(19) United States
(12) Patent Application Publication
(10) Pub. No.: US 2003/0050274 A1
(43) Pub. Date: Mar. 13, 2003

(54) Composition containing S-adenosylmethionine for treating or preventing insulin resistance syndrome

(76) Inventor: Hong-Kyu Lee, Seoul (KR)

Correspondence Address: David A. Einhorn, Esq., Anderson Kill & Olick, P.C., 1251 Avenue Of the Americas, New York, NY, 10020, US

(21) Appl. No.: 09/927,839

(22) Filed: Aug. 10, 2001

(30) Foreign Application Priority Data
May 17, 2001 (KR) 2001-27026

(51) Int. Cl. A61K 31/7076

(52) U.S. Cl. 514/46

(57) ABSTRACT

Described in the present invention is a pharmaceutical composition for treating or preventing insulin resistance syndrome such as obesity and diabetes in a mammal, which comprises an effective amount of S-adenosylmethionine as an active ingredient together with a pharmaceutically acceptable carrier.

### FIELD OF THE INVENTION

The present invention relates to a pharmaceutical composition for treating or preventing insulin resistance syndrome such as obesity and diabetes in a mammal, which comprises an effective amount of S-adenosylmethionine as an active ingredient together with a pharmaceutically acceptable carrier.

### BACKGROUND OF THE INVENTION

Insulin resistance syndrome is a loosely-defined clinical entity, which generally refers to the syndrome including hypertension, obesity, dyslipidemia, diabetes, cardiovascular disease and glucose intolerance in which resistance against insulin's activity of lowering a blood glucose level is a common feature. Because insulin resistance usually develops long before the diseases appear, identifying and treating insulin-resistant patients has potentially great preventive value. Insulin resistance should be suspected in patients with a history of diabetes in first-degree relatives; patients with a personal history of gestational diabetes, polycystic ovary syndrome or impaired glucose tolerance; and obese patients, particularly those with abdominal obesity. Present treatment consists of sensible lifestyle changes, including weight loss to attain healthy body weight, 30 minutes of accumulated moderate-intensity physical activity per day and increased dietary fiber intake. Pharmacotherapy is not currently recommended for patients with isolated insulin resistance. It is reported that the insulin resistance syndrome is accompanied by a decrease in the DNA copy number present in mitochondria (LEE, H. K. et al., Diabletes Res Clin Pract., 42:161-167, 1998)

S-adenosylmethionine (SAMe), an intermediate formed during the synthesis of methionine, acts as a methyl group donor in every living body: It provides methyl groups in methylation reactions of intracellular materials including phospholipids, methylating proteins, CpG island domain in DNA, and adrenaline, dopamine and serotonin functional materials. Any dysfunction of such methyl group transfer by S-adenosylmethionine adversely affects various intracellular biomechanisms responsible for such matters as the gene expression and cell membrane fluidity. For instance, inhibition or activation of a gene depends on the methylation or demethylation process of DNA. In this regard, it has been reported that S-adenosylmethionine is effective for the treatment of melancholia and also for improving movement ability of rats exhibiting symptoms similar to Parkinson's disease (Osman E. et al., Aliment Pharmacol Ther.(ENGLAND), 7:21-28, 1993; Crowell B. G. Jr. et al., Behav Neural Biol., 59:186-193, 1993; and Bottiglieri T. et al., Drugs, 48:137-152, 1994).

The present inventors have endeavored to develop a novel pharmaceutical use of S-adenosylmethionine, and have discovered that S-adenosylmethionine is effective for the treatment of insulin resistance syndrome.

### SUMMARY OF THE INVENTION

Accordingly, it is an object of the present invention to provide a pharmaceutical composition containing S-adenosylmethionine for treating or preventing insulin resistance syndrome.

In accordance with one aspect of the present invention, there is provided a pharmaceutical composition for treating or preventing insulin resistance syndrome in a mammal, which comprises an effective amount of S-adenosylmethionine as an active ingredient and a pharmaceutically acceptable carrier.

### BRIEF DESCRIPTION OF THE DRAWINGS

The above and other objects and features of the present invention will become apparent from the following description of the invention, when taken in conjunction with the accompanying drawings, which respectively show:

FIG. 1 : weight changes observed for the OLETF (Otsuka Long Evans Tokushima Fatty) rats administered with S-adenosylmethionine and non-S-adenosylmethionine, as well as the LETO (Long Evans Tokushima Otsuka) rats of the control group;

FIG. 2 : glucose load test results obtained for the OLETF rats administered with S-adenosylmethionine and non-S-adenosylmethionine, as well as the LETO rats of the control group; and

FIG. 3 : euglycemic hyperinsulinemic clamp test results obtained for the OLETF rats administered with S-adenosylmethionine and non-S-adenosylmethionine, as well as the LETO rats of the control group.

### DETAILED DESCRIPTION OF THE INVENTION

S-adenosylmethionine is capable of increasing insulin sensitivity, i.e., lowering resistance against insulin's activity in a mammal, and thus, it exerts inhibitory as well as therapeutic effects on insulin resistance syndromes, e.g., obesity and diabetes. Further, S-adenosylmethionine exhibits no toxicity when it is orally administered to a rat at a dose of 500 mg/kg.

A pharmaceutical formulation may be prepared in accordance with any of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxy-benzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of S-adenosylmethionine may range from about 1 to 30 mg/kg body weight, preferably 5 to 20 mg/kg body weight, and can be administered in a single dose or in divided doses.

However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

As described above, S-adenosylmethionine can be used as an effective, non-toxic pharmaceutical agent for treating or preventing insulin resistance syndromes, e.g., obesity and diabetes.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### EXAMPLE 1

### Effect of S-Adenosylmethionine on the Insulin Secretion Function and Insulin Sensitivity

14 six-week-old OLETF (Otsuka Long Evans Tokushima Fatty) rats (Otsuka Research Lab., Tokushima, bred for use as a non-insulin-dependent diabetes mellitus model) were evenly divided into two injection groups. The rats of two groups were administered into the abdominal cavity either with S-adenosylmethionine (SAMe(+) group) or with a vehicle containing L-lysine (SAMe(−) group) at a daily dose of 15 mg/kg body weight for a duration of up to 19 weeks. 7 six-week-old LETO (Long Evans Tokushima Otsuka) rats (Otsuka Research Lab., Tokushima, normal rats) were used as a normal control group (Control group). The rats of each group were examined for their weight changes, and checked by way of conducting glucose load tests and euglycemic hyperinsulinemic clamp tests to measure the effect of S-adenosylmethionine on the insulin secretion and insulin sensitivity.

Test data were analyzed to represent each test result in the form of mean±standard error mean. Differences in weight changes among the groups were analyzed by ANOVA with Least Significance Difference using SPSS/PC 10.0, and the differences in glucose load and clamp test results, by the Kruskall-Wallis test and checked using the Mann-Whitney probation method. The significance level was set at a p value of 0.05 or below.

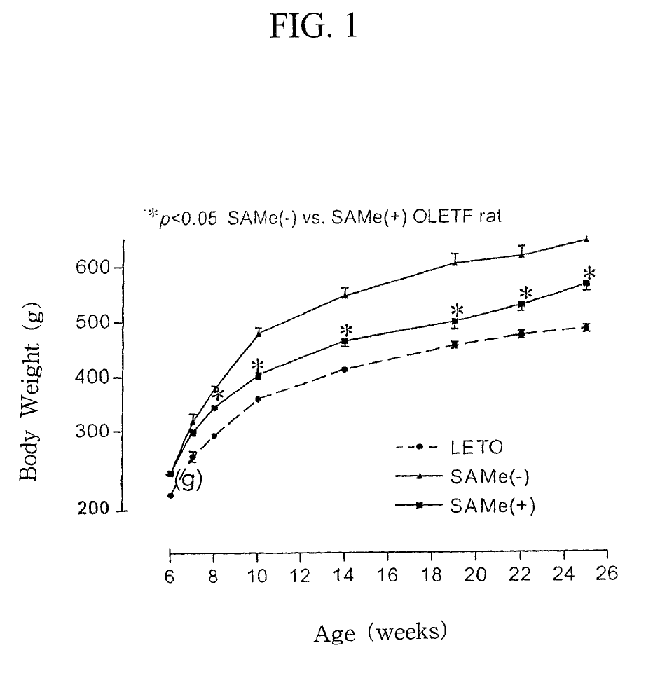

(1-1) Weight Change At 0, 1, 2, 4, 8, 13, 16 and 19 weeks after the administration had begun, the weight changes of the rats of the three groups were recorded. The result is shown in Table I and FIG. 1.

TABLE I

| | Body Weight (g) in accordance with the Administered | | | | | | | |
|---|---|---|---|---|---|---|---|---|
| | Week Number | | | | | | | |
| Group | 0 | 1 | 2 | 4 | 8 | 13 | 16 | 19 |
| LETO rats | | | | | | | | |
| Control group | 178 ± | 251 ± | 290 ± | 358 ± | 411 ± | 455 ± | 473 ± | 484 ± |
| | 1.9 | 9.4 | 2.4 | 3.0 | 4.7 | 5.9 | 6.7 | 8.3 |
| OLETF rats | | | | | | | | |
| SAMe(+) | 220 ± | 299 ± | 343 ± | 403 ± | 464 ± | 498 ± | 528 ± | 565 ± |
| group | 2.8 | 8.8 | 4.7 | 6.6 | 10.8 | 14.0 | 1.8 | 12.4 |
| SAMe(−) | 219± | 317 ± | 377 ± | 478 ± | 547 ± | 603 ± | 617 ± | 644 ± |
| group | 3.7 | 13.6 | 5.8 | 10.1 | 13.7 | 17.7 | 16.3 | 15.3 |

As can be seen from Table I and FIG. 1, the ratio of weight gain was significantly lower for the SAM(+) group, as compared with the SAM(−) group. The rate of weight gain of the Control group was significantly lower than those of the OLETF rats for the whole test period.

The above result suggests that S-adenosylmethionine is effective in inhibiting fatness caused by an increased blood glucose level.

(1-2) Glucose Load Test

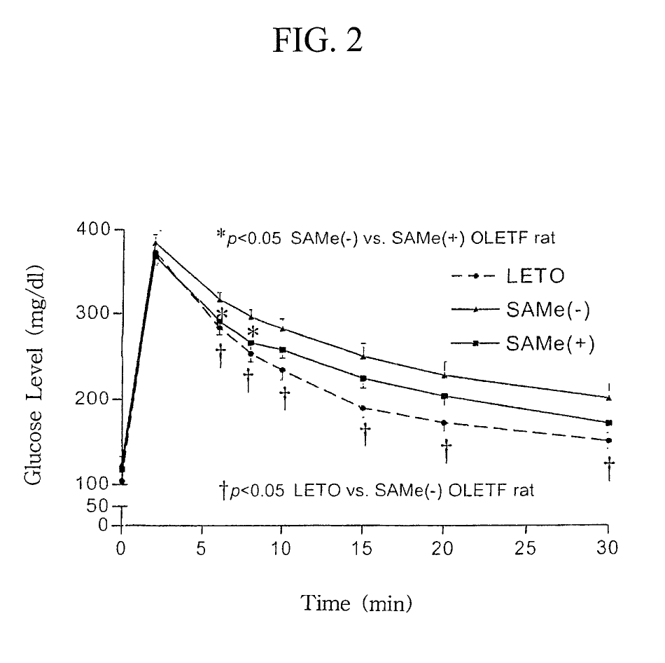

On the 19th week after the administration had begun, the rats were starved for 6 hours and a 35% glucose solution (0.5 g glucose/kg body weight) was bolus-infused into each rat through the tail artery. At 0, 2, 6, 8, 10, 15, 20 and 30 minites after the infusion, a 200 μl blood sample was taken from the tail artery of each rat, centrifuged and the plasma supernatant was separated. The blood glucose level was determined by employing YSI 2300. The result is shown in Table II and FIG. 2.

TABLE II

| | Blood Glucose Level (mg/dl) | | | | | | | |
|---|---|---|---|---|---|---|---|---|
| | in accordance with the Time (min) after the Infusion | | | | | | | |
| Group | 0 | 2 | 6 | 8 | 10 | 15 | 20 | 30 |
| LETO rats | | | | | | | | |
| Control group | 104 ± | 373 ± | 283 ± | 252 ± | 234 ± | 190 ± | 172 ± | 150 ± |
| | 2.7 | 13.2 | 8.6 | 9.3 | 11.2 | 11.1 | 9.9 | 9.1 |
| OLETF rats | | | | | | | | |
| SAMe(+) | 118 ± | 368 ± | 291 ± | 265 ± | 257 ± | 224 ± | 204 ± | 172 ± |
| group | 4.3 | 10.2 | 6.8 | 7.8 | 9.6 | 11.3 | 10.8 | 11.8 |
| SAMe(−) | 125 ± | 384 ± | 317 ± | 296 ± | 282 ± | 249 ± | 228 ± | 201 ± |
| group | 8.1 | 10.5 | 8.1 | 8.0 | 12.1 | 15.0 | 15.7 | 19.2 |

As can be seen from Table II and FIG. 2, the blood glucose levels measured at 6 and 8 minutes after the infusion were significantly lower for the SAM(+) group, as compared with the SAM(−) group, while the blood glucose level of the Control group was significantly lower than those of the OLETF rats for the whole test period.

In addition, the glucose loss rate ( % /min) was calculated from the inclination of log-converted values of glucose concentrations measured during 2 to 15 minutes after the infusion. The glucose loss rates of the SAM(+) and SAM(−) groups were similar at 3.69±0.29 and 3.23±0.32%/min, respectively, while that of the Control group was much higher at 5.13±0.29%/min. The areas obtained by integrating the blood glucose level curves for the measurement time period were 234±9.6, 259±13.1 and 213±9.4 (mg/dl).min, for the SAM(+), SAM(−) and Control groups, respectively, showing no significant differences among them.

(1-3) Euglycemic Hyperinsulinemic Clamp Test

On the 19th week after the administration had begun, the rats were starved for 6 hours, and a 200 μl blood sample was taken from the tail artery of each rat to determine the basal glucose and insulin levels. Insulin (Novolin-R, 0.24 U/ml) was continuously infused into the tail artery at a rate of 12 mU/kg/min for 120 min to maintain hyperinsulinemia, and every 20 minutes, 200 μl blood samples were taken therefrom to measure the plasma insulin level. Further, every 10 minutes after the infusion of insulin, 50 μl blood samples were taken therefrom to measure the plasma glucose level, while a 25% glucose solution was continuously infused into another tail artery at such a rate to maintain the measured basal glucose level. The plasma glucose and insulin levels would reach steady states during the period of 60 to 120 min after the infusion of insulin, and thus this period was selected as a clamp test period.

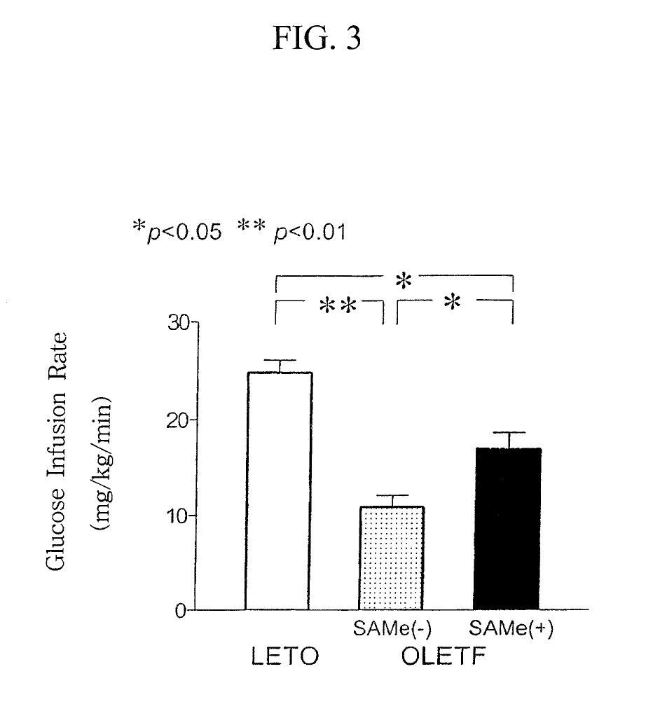

Based on the fact that the amount of glucose infused to maintain the steady basal level is equal to the amount of glucose consumed in the body, insulin resistance of each group was determined from the mean glucose infusion rate (mg/kg/min) during the clamp test period. The SAM(+) group has a mean glucose infusion rate of 16.7±1.70 mg/kg/min; the SAM(−) group, 10.9±1.19 mg/kg/min; and the Control group, 24.8±1.30 mg/kg/min, as shown in FIG. 3.

From the above result, it is confirmed that S-adenosylmethionine effectively lowers the insulin resistance and thus, it can be advantageously used for treating or preventing diabetes.

### EXAMPLE 2

### Toxicity of Orally Administered S-Adenosylmethionine

8 six-week-old, specific pathogen-free SD-based rats were evenly divided into four groups, which were orally administered with S-adenosylmethionine suspended in a 0.5% methylcellulose solution at a dose of 50, 100, 200 and 500 mg SAMe/kg body weight, respectively. Then, the rats were observed for 10 days for signs of adverse effects or death according to the following schedule: 1, 4, 8, and 12 hours after the administration. Every 12 hours thereafter, the weight changes of the rats were recorded to examine the effect of S-adenosylmethionine. Further, on the 10th day, the rats were sacrificed and the internal organs were visually examined.

All the rats were alive at day 10 and S-adenosylmethionine showed no toxicity at a dose of 500 mg/kg. The autopsy revealed that the rats did not develop any pathological abnormality, and no weight loss was observed during the 10 day test period. Accordingly, it was concluded that S-adenosylmethionine is not toxic when orally administered to an animal.

Praparation 1: Preparation of Syrup

A syrup was prepared using the following ingredients, containing S-adenosylmethionine in an amount of 2 wt/vol %:

| | Quantity (g/syrup) |
|---|---|
| S-adenosylmethionine | 2 |
| Saccharin | 0.8 |
| Polysaccharide | 25.4 |
| Glycerin | 8.0 |
| Flavor | 0.04 |
| Ethanol | 4.0 |
| Sorbic acid | 0.4 |
| Distilled water | necessary amount |

S-adenosylmethionine, a portion of saccharin, and polysaccharide were dissolved in warm distilled water, and cooled, while glycerin, the remaining portion of saccharin, flavor, ethanol and sorbic acid were dissolved in distilled water. The two solutions were mixed and distilled water was added thereto up to a total volume of 100 ml, to obtain a syrup preparation.

Praparation 2: Preparation of Tablet

A tablet containing 15 mg of S-adenosylmethionine was prepared using a mixture of the following ingredients:

| | Parts |
|---|---|
| S-adenosylmethionine | 250 |
| Lactose | 175.9 |
| Potato starch | 340 |
| Colloidal sillicic acid | 32 |
| Talc | 50 |
| Magnesium stearate | 5 |
| 10% Gelatin solution | necessary amount |

S-adenosylmethionine, lactose, a portion of potato starch and colloidal sillicic acid were mixed and a 10% gelatin solution was added thereto. The mixture was pulverized and passed through a 14 mesh filter. The powder was dried, and the remaining portion of potato starch, talc and magnesium stearate were added thereto to obtain a mixture, which was then processed into tablets by a conventional method.

Praparation 3: Preparation of Injectable Solution

An injection preparation containing 10 mg/100 ml of S-adenosylmethionine was prepared using the following ingredients:

| | Parts |
|---|---|
| S-adenosylmethionine | 1 |
| Sodium chloride | 0.6 |
| Ascorbic acid | 0.1 |
| Distilled water | necessary amount |

S-adenosylmethionine, sodium chloride and ascorbic acid were dissolved in distilled water to a total volume of 100 ml. The resulting solution was put in a bottle and heated at 200° C. for 30 min. to obtain a sterile injectable solution preparation.

While the embodiments of the subject invention have been described and illustrated, it is obvious that various changes and modifications can be made therein without departing from the spirit of the present invention which should be limited only by the scope of the appended claims.

What is claimed is:

1. A pharmaceutical composition for treating or preventing insulin resistance syndrome in a mammal, which comprises an effective amount of S-adenosylmethionine as an active ingredient and a pharmaceutically acceptable carrier.

2. The composition of claim 1, wherein the insulin resistance syndrome is obesity or diabetes.
